(19) United States
(12) Patent Application Publication
(10) Pub. No.: US 2023/0024246 A1
(43) Pub. Date: Jan. 26, 2023

(54) HELICOIDAL BODY FOR CUTTING, REMOVING AND CLEANING AND PRODUCTION METHOD

(71) Applicant: Mario Valderrama Chaparro, Bogota (CO)

(72) Inventor: Mario Valderrama Chaparro, Bogota (CO)

(21) Appl. No.: 17/758,402

(22) PCT Filed: Dec. 4, 2021
(86) PCT No.: PCT/IB2021/061342

§ 371 Date: Jul. 5, 2022

(30) Foreign Application Priority Data
Dec. 7, 2020 (CO) NC2020/0015320

(51) Int. Cl. A61F 13/38; A61F 11/00

(52) U.S. Cl. CPC A61F 13/38; A61F 11/006; A61F 13/385

(57) ABSTRACT

Helical body with blades for cutting, stirring, removing and cleaning in solids and closed and open tubular shapes. The blades create more points to cut and contact with less material and remove what they cut and withdraw in the same movement. In ear cleaning they reduce the risk of pushing in the cerumen. The helicoid to remove earwax is manufactured with the same processes and machines of the art of swabs, adding two steps, preforming and forming. This one, by placing halves of helicoid molds in bands or cylinders where, by pressure, a vacuum, and heat, they form the blades. The machine produces quantities per minute similar to the current ones. Uses of the invention are possible in cutting and removal of rocks, in mechanical tools for working on metals or hard materials, in medicine for removal and extraction in ducts, veins and arteries, in personal use and domestic utensils where blades replace fibers.

### FIELD OF INVENTION

The invention generally relates to the field of means and machines for cutting, removing, withdrawing, and cleaning solids or contours with surfaces of a generally, closed or open, tubular structure. The invention has application in the removal of rocks or solids, in cleaning pipes and spaces with a generally concave shape, and in specifically, in the removal and withdrawing of earwax. The invention refers to the way of manufacturing helicoids to remove earwax and similar applications.

### STATE OF THE ART

Means and tools for cutting, removing, and cleaning generally consist of a part that removes but does not remove the cut material, which is done by another action. These means are used in the excavation of tunnels of different sizes and purposes and in excavations to place pipes and other elements.

The same happens with the cleaning of pipes in the food, chemical and mixture industries, after each time or completion of the process. Cleaning methods in the art consist of hot and pressurized chemicals, held long enough to ensure removal and cleanliness. The substances are polluting and are generally dumped into riverbeds. A physical element that contacts the internal surfaces of these ducts, whether closed or open, seems convenient, and with the injection of less polluting substances, cleaning is fast and reliable.

Domestic and personal cleaning items, such as bristle brushes, use elements that stir but do not remove, they are generally made of hard plastic fibers that are difficult to degrade when discarded and by being small fibers, can cause damage if they are swallowed. With the helicoid, a single piece can be formed that is more efficient and less polluting than the one made of fibers. It also allows the cleaning of earwax to be made with softer contact surfaces and preventing penetrating to damage delicate parts of the ear.

Cotton swaps for cleaning ear wax and other uses that have an ovoid shape, are attribute several drawbacks to them:

1. They are ecologically offensive because the plastic grip is not easily degradable, although it has been replaced by other degradable materials such as cellulose.

2. Its conical shape pushes the earwax into the ear, and its flat surface can remove hair from the canal, so it is recommended not to use it. This also applies to applying and cleaning delicate areas.

3. They can damage delicate inner parts of the ear by being hard and not preventing the depth to which the user can push it in.

Numerous applications are possible based on the invention. Either can be used manually or motorized to clean external and internal parts of cavities, pipelines, and the like and closed and open concave areas. For example,

a) Cutting and removal of materials and tools for working on metals or hard materials.

b) The current technique in tunneling is done with a large disk that has cutting pieces and reinforcements more resistant. The disc, with a diameter equal to the final cavity, rotates, cuts and crushes rocks by layers of the entire surface, which requires a very resistant structure and potency. If this surface has parts of hard rock and gravel or less hard rocks, the turning and shearing stress over the entire diameter is equal to the one needed for the hardest part. It does not provide a choice to varying the cutting if the surface is not homogeneous.

c) Also, if there is failure in a part of the disk it is necessary to halt the entire operation.

If cutting means composed of separate pieces such as the helicoid are used, mounted in a system where each cutting part rotates together or independently, it may be possible to position them so that they cover the same diameter as that obtained with a large disc.
1) Each cutting helicoid can act according to the condition of the material to be cut
2) Remove only a part of the entire surface to act around the hardest part to weaken it and remove it in parts.
3) Drill with different speeds of each helicoid.
4) If one cutting element fails, the others can continue to operate.
5) If the direction of rotation is changed, the helical body pushes material into a cavity. In excavations, holes are drilled to place explosive material, which are then covered for greater effectiveness. The helical body, rotating in one direction, drills cavities and in the opposite direction pushes material to cover them.
6) A system of sensors of hardness, resistance to cutting can be installed that are transmitted to an Al unit so that it adjusts each cutting element, according to the conditions of the cut element.

d) Applications in Medicine and the like, surgical devices to clean veins and arteries, prostate surgery, and other cavities. In nano helical bodies, movement can be achieved not by electric motor but by chemical action or by cells, which exist in art.

e) Applications in utensils for domestic or personal use such as earwax cleaning, where contact with the ear is soft and the base can be widened to control that it does not reach delicate parts.

f) Other possible applications are cleaning probes, robots to move inside the pipe, motorized with motors in the helical or remote.

g) The shape of the helicoid blades can be optimized for the function to be performed, calculated quantitatively and their performance tested before construction. In the art there are applications for both and 3D printing for tests, in a short time and at low cost.

The following inventions propose new forms of helical shape to remove and clean earwax, with characteristics different from those of the invention.

USD545431S—Spiral Grooved Head for an Ear Cleaning Swab by NAMI DESIGN LLC, shows spirals defined in conical shape, with flat cutting and contact edges, it does not indicate the geometric shape of the remover and cleaning plane and therefore the apex of the cone is shaped oval that is not optimal to remove and clean the innermost part. It can have a pushing effect, like the ovoid ones. It does not show how to build it.

It shows spirals defined in cylindrical and ovoid shapes, with flat edges and contact. The apex of the cone is oval in shape, which is not optimal for removing and cleaning the innermost part. It will tend to push in the earwax, like the ovoid shaped ones. It does not say how to build it.

Patent 2003/0135228 published as US2008142385A1; US2009173650A1. It proposes an ovoid-shaped earwax cleaning swab where each spiral starts at one end of the handle and ends loosely. The handle ends in a point to prevent deep cleaning, which can damage the eardrum.

The following inventions to remove ears wax differ to the proposed invention because they are not helicoid, not conical, do not show how to build it or are not disposable, U.S. Pat. Nos. 6,033,417, 5,632,756, 5,374,276, 3,923,061, JP2017511158A, US20130304103A1, U.S. Pat. Nos. 7,658,745B2, 9,867,738B2, 9,867,738B2, US209125588A1, TWM484411U.

The following inventions propose new forms of helicoid to cut and remove several kinds of materials, with characteristics different from those of the invention, they do not include modifying the shape of the cutting plane to optimize the operation, they are elongated spirals, not suitable for removing material chopped up. Some propose reinforcements, articulated sections, but not integrated or the components to lighten, adjust to electronics and Artificial Intelligence means. U.S. Pat. No. 3,715,788A, CA435662A, CA709213A, CA2486839C, A209330A, JP5023628B2, KR101369580B1, GB2342372A, GB2558172A.

### GENERAL DESCRIPTION

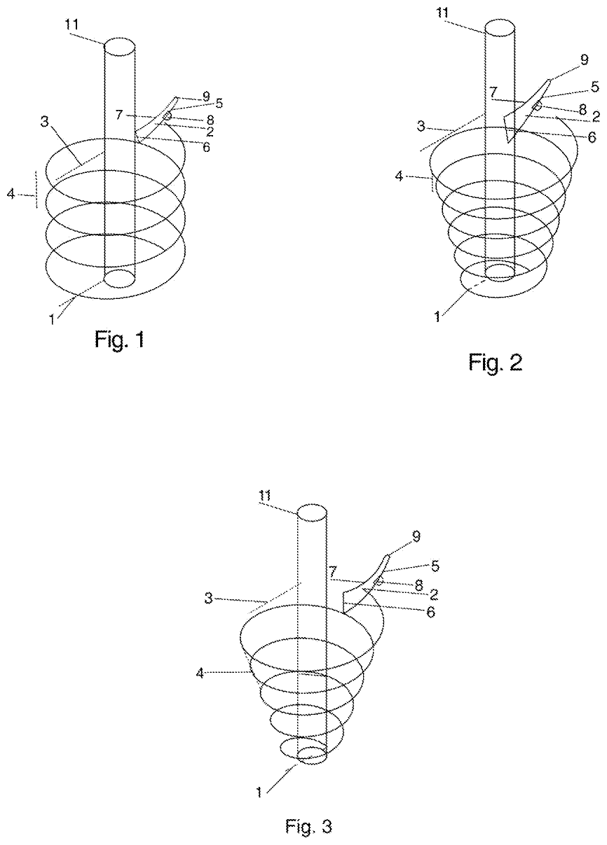

FIG. 1 shows a schematic perspective view of the cylindrical helicoid body with the grip (11) and the cross section (2) of the plane that forms four blades (27) that cut, remove, and withdraw material and other characteristics.

FIG. 2 shows a schematic perspective view of the conical helical body with six blades (27).

FIG. 3 shows a schematic perspective view of the conical helical body with five blades (27).

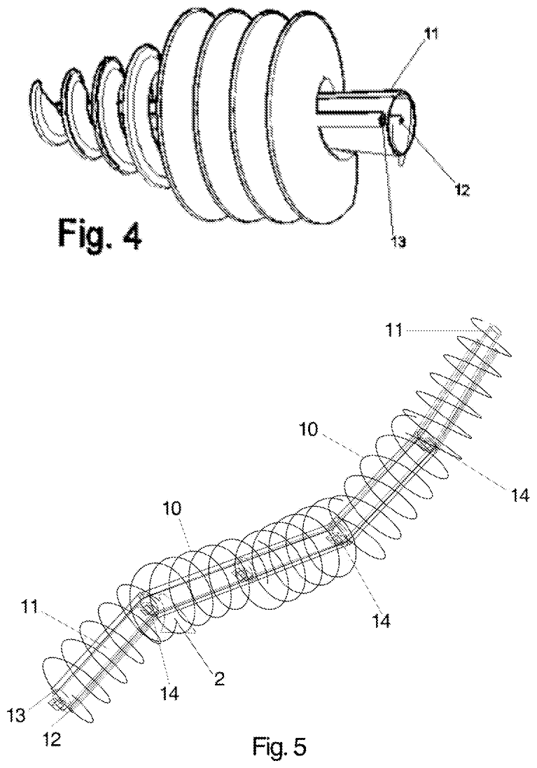

FIG. 4 shows a perspective view of the helical body with two ducts located along its grip, internally or on its sides.

FIG. 5 shows a perspective view of the helical body made up of several sections (10), with unions or couplings (14) on their axes.

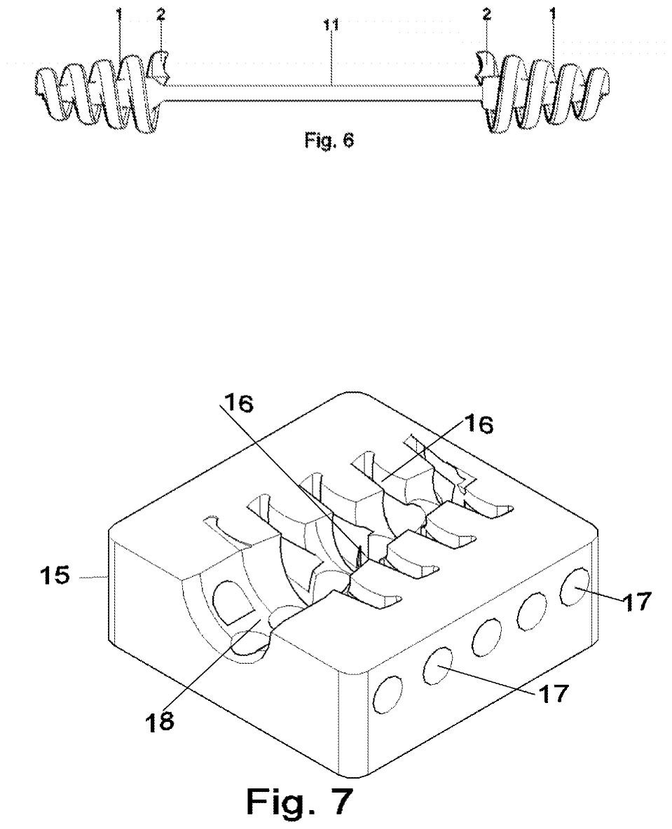

FIG. 6 shows a perspective view of a helicoid body (1) to remove and clean earwax with helicoids at the ends of the grip (11).

FIG. 7 shows a perspective view of half the mold to manufacturing the helicoid for cleaning ear wax.

FIG. 8 shows a perspective view of a box and lid (15A) that houses half of the mold (15) of the helical body, closed at the bottom and open at the top, creating a cavity (20) where in its inside space the half mold of the helicoid is housed.

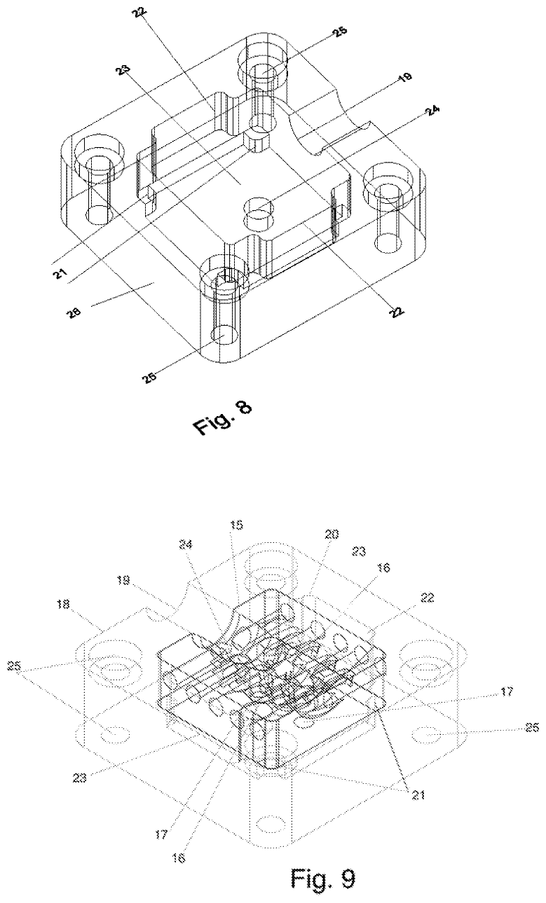

FIG. 9 shows a perspective view of the box assembly (15A) housing half of the helicoid mold (15), with the characteristics described in FIGS. 7 and 8.

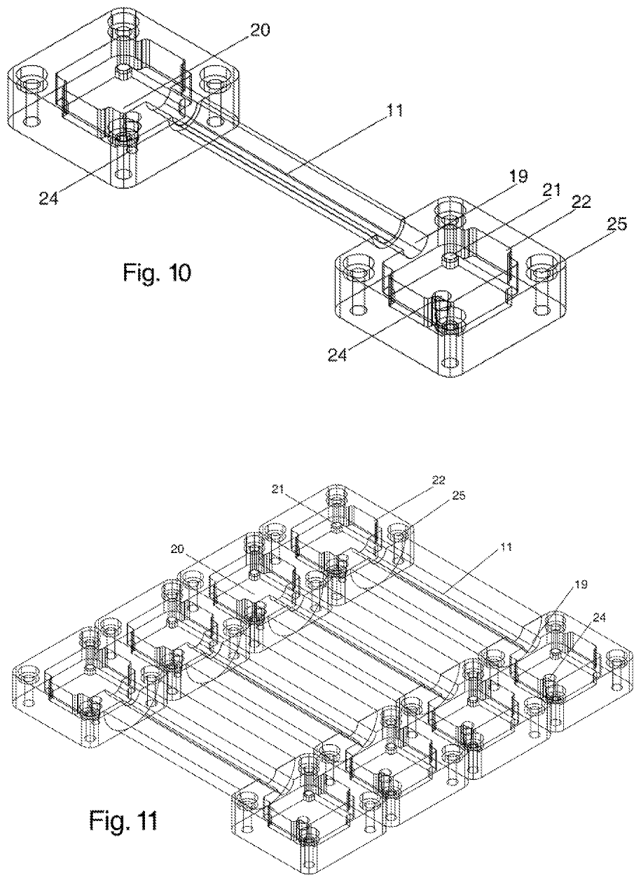

FIG. 10 shows a perspective view of the set of boxes to house the half-molds that form the helical body

FIG. 11 shows a perspective view of a group of 4 boxes to house half molds of the helical body.

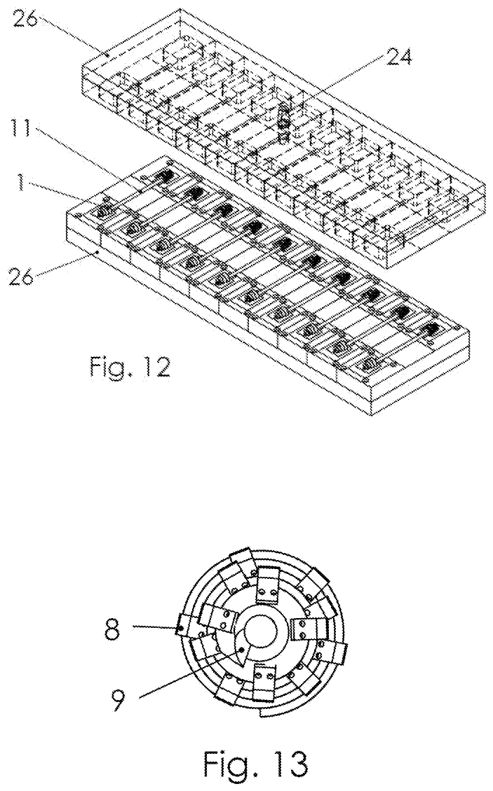

FIG. 12 shows a perspective view of a box frame (26) to place ten boxes on a band where ten helicoid half-molds are accommodated. It shows a second housing for boxes aligned and raised above the first with ten cavities to house ten helicoid half-molds to form the helicoid body.

FIG. 13 shows a plant view of a helicoid for cutting excavation materials such as rocks, with the shaft (11), external edges of blades (5) and reinforcements (8) secured to them.

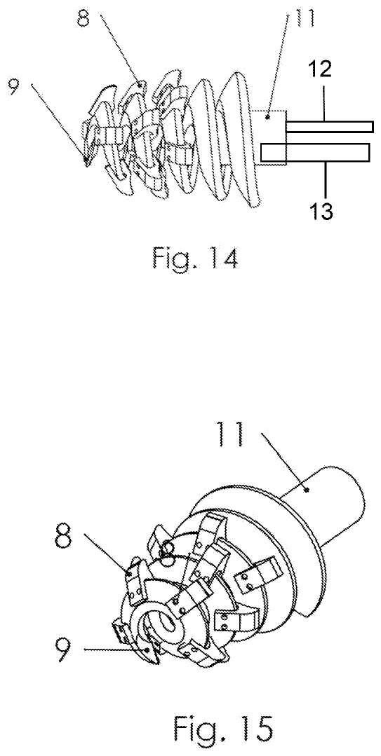

FIG. 14 shows a side view of a helicoid for cutting excavation materials such as rocks, with the grip (11), outer edges of blades (5) and reinforcements (8) secured to them, injector duct (12) and material extraction duct (13).

FIG. 15 shows a perspective view of a helicoid for cutting excavation materials such as rocks, with the shaft (11), external edges of blades (5) and reinforcements (8) secured to them.

### DETAILED DESCRIPTION OF THE INVENTION

The following figures illustrate generally the proposed invention but are not limited to these illustrations.

FIG. 1 shows a schematic perspective view of the cylindrical helicoid body (1) with the cross section (2) of the plane that moves along the axes to form four blades (27) that cut, remove, and withdraw material in the same movement by turning it in the proper direction. It also shows the length of the equal radii of the base (3) and the apex of the helicoid body (1), the distance (4) between the blades (27), base (6) of the blades, the shape and curvature of the edge internal blade (7) and external blade edge (5) of the blade where reinforcements (8) can be located, the cutting tip (9) that can also be reinforced and the grip (11). The number of twists, the distance between the base and the apex vary according to the specific application and the material of which it is built.

FIG. 2 shows a schematic perspective view of the conical helical body (1) with the cross section (2) of the plane that moves along the axes to form six blades (27). It also shows the characteristics described in FIG. 1.

FIG. 3 shows a schematic perspective view of the conical helical body (1) with the cross section (2) of the plane that moves along the axis to form five blades (27). It also shows the characteristics described in FIG. 1.

Due to its circular shape, the top and bottom plant view of the helicoid will show continuous concentric circles that can be of different diameter and number.

FIG. 4 shows a perspective view of a helical body with two ducts located along its grip, internally or on its sides. An injector duct (12) injects the appropriate substance according to the material that is cut and removed, for example, water in mining, soaps and removers in cleaning ducts in industrial processes, chemical or biological substances in medicine such as coagulant and anti-inflammatory in prostate operation and the like. The second extractor duct (13) removes removed or cut material and the injected substance.

FIG. 5 shows a perspective view of a helical body made up of several sections (10), with joints or couplings (14) on their axes so that they are oriented and rotate at the same angles and curves as the object where it is going to be cut, remove and clean and orient it in the desired direction. Ducts indicated in FIG. 4 can be added to this body. Each section can rotate independently and at different speeds by means of cables or motors (not shown) at each section joint and have lights and sensors that are in the art. The helical sections can become traction elements to move the assembly forward or backward. It can be connected to a system with instructions and create a cutting and removal body that accumulates information and acts with Artificial Intelligence.

FIG. 6 shows a perspective view of a helicoid body (1) to clean earwax and other uses with a helicoid at the ends of the grip (11) with blades of which the cross section of the plane (2) is shown, which gives the desired shape to the blades. Although not listed, this helical body has the characteristics described in FIG. 1.

With the same amount of material used in the ovoid shape, this helical body creates a larger contact area and smoother, more cutting angles, removes, removes wax or any other material from the duct with less pressure, the outer edges of blades (5) of the blades contact smaller points and on a different surface than a contact element with only an ovoid general surface. The curvature of the blade helps to remove and withdraw material more effectively without the risk of pushing it; the base wider than the apex reduces the risk of damaging internal parts of the ear.

FIG. 7 shows a perspective view of half the mold of a helical body. The two halves are not identical, each half is the product of the longitudinal cut of the helical body along the grip. The cavities (16) of the blades and the injection holes (17) that communicate 3 sides of the mold with the cavity of the box that is described in FIG. 8 and with the cavities (16) where the blades of the helicoid body are formed. It shows the concave area (18) of the mold that forms part of the grip (11) and that contacts the concave area (19) of the box (28) described in FIG. 8.

FIG. 8 shows a perspective view of a box (28), which are the same, designed to house any of the half of the molds of the helicoid in its interior space and that can be changed to change only half of each mold and manufacture with it. same process different shapes and sizes depending on the use for which it is intended. The box (28) has a concave area (19) to house part of the grip, a cavity (20) to house half of the helicoid mold and grips (21) to leave space for air circulation below the mold. This cavity (20) communicates with 2 lateral spaces (22) to extract air from the sides of the mold shown in FIG. 7. In the lower part (23) of the box there is a connector hole (24) to fix a coupling cooperant to connect the box to an air extraction mechanism that produces negative pressure in the box and mold. It also shows 4 holes (25) to screw the box to a grip that allows accommodating several boxes as shown in FIG. 12 to form the helicoid body.

FIG. 9 shows a perspective view of the box with the same characteristics described in FIG. 8 holding half of the helicoid mold described in FIG. 7.

FIG. 10 shows a perspective view of the 2 boxes (28) and the grip (11) that form half of the mold of the helical body with its concave area spaces (19) to house part of the grip (11), the cavity (20) where the helicoid half mold is housed, described in FIG. 8, with the characteristics described in the previous figures.

FIG. 11 shows a perspective view of a group of 4 molds for housing half helicoid molds that form half of the helicoid body. Each one with a connector hole (24) and communication to a system to produce vacuum. It shows the characteristics described in the previous figures.

FIG. 12 shows a perspective view of a frame (26) for placing ten boxes on a band where ten half molds of helicoidal bodies (1) and in each one a preformed helicoidal body with its grip (11) are accommodated. It shows a second grip aligned and raised above the first one with ten cavities to house ten helicoid half-molds (not shown) and grips. Both grips with a connector (24) to create a vacuum in the internal space that communicates with the cavities of each box and of the half-molds that are housed in them.

FIG. 13 shows a plant view of a helicoid for cutting excavation materials such as rocks, with the grip (11), external edges of blades (5) and reinforcements (8) secured to them.

FIG. 14 shows a side perspective view of a helicoid for cutting excavation materials such as rocks and mining with the grip (11), external edges of blades (5) and reinforcements (8) secured in them, with injector duct (12) and material extraction duct (13). This modality, combined with the one described in FIG. 5, can be used in mining to inject the appropriate material and remove the removed materials for the separation process. It allows to follow the vein and extract only the vein, with minimal removal of adjacent material and reduce the negative effect on the ecology. Visors, lights, testers, etc. can be added to this modality as needed. exist in art.

FIG. 15 shows a perspective view of a helicoid for cutting excavation materials such as rocks, with the grip (11), external edges of blades (5) and reinforcements (8) secured to them.

### Manufacture Process

The new helical body for removing and withdrawing ear wax can be built with the same known machines, if part of the process for creating the helical blades is modified, as described.

Step 1—Set up the material from the manufacturer, grip and helicoid material, this one can have in several forms:

1. Material with which the helicoid is manufactured cotton, cellulose or other material in the art, appropriate to manufacture the helicoid body, which can have various forms:
a) flat band 8 to 10 mm wide, 1 to 2 mm thick.
b) inclined plane band 8 to 10 mm wide, where one side is 3 to 4 mm thick and decreases to 1 to 2 mm towards the other side.
c) cylindrical strip, like a thread, with a diameter of 1 to 2 mm.

In any of these forms the material is pre-cut or continuous. In this case the preforming step includes the cutting of each part when spinning.

2. Preformed grip, made of impregnated paper, cellulose, wood, 50 to 60 mm long and 2 to 3 mm in diameter. It can have different lengths and diameters depending on the application for which it is intended.

Step 2—Add emollient to each end of the grip to help giving and keeping shape, remove fiber particles that can come off inside the ear, by dispersion in cold, hot, steam, about 0.025 ml.

Step 3—Preform the helicoid body by wrapping 0.5 to 1 gram of cotton, cellulose, or other material with suitable characteristics (soft, absorbent, non-particles) around the two ends of the grip.

This winding results in a conical helical body with diameters at the base of 5 to 6 mm and at the upper end of 2.5 to 5 mm, which are 25 to 30% larger than the final size of the helical body for cleaning wax. These measures are modified depending on the application to which it is intended. The pre-shape can be made in several ways:
a) Place the grip (11) in a band or cylinder, which has a cavity with its shape and means to make it rotate.
b) Mold a flat band of material such as cotton in the middle of a longitudinally cut mold that has the shape of the helicoid in low relief, hold independently of the grip (11). This half of the mold is engraved in a block 20 to 23 mm square and 6 to 13 mm high and varies according to the application for which it is intended.

The movement of the band or the cylinder causes the material to be rolled to come closer and remains in contact with each end of the grip (11) and in proximity to the cavity that has the half mold of the helicoid engraved in low relief. At the ends of the grip (11) the material, such as cotton, is hold, rotated and rolled over the half of the mold, forcing it to take the shape of the half mold of the helicoid body. At each end 0.02 to 0.03 grams of the material are wound. The movement of the band or the cylinder brings the pre-formed helical body to a storage space.
c) Roll up a cylindrical strip of material at each end of the grip, giving more winding time at the base of the cone and moving towards the smaller diameter. A cone-roll with more material at the bottom will result.
d) Mold a cylindrical strip by rolling against half a mold while the material source moves longitudinally to the axis of the mold.

Step 4—Place the preformed helicoid bodies in a container that is close to the band or cylinder that gives the final shape.

Step 5—Arrange the preformed helicoid bodies in the grip that is on a band and that contains the halves of the mold of ten helicoid bodies, band that moves as it is currently done in the art.

Step 6—Give final shape in cycles. Ten boxes with half helical body mold and grip are accommodated in a frame on a moving belt. The grip has in its lower part a connecting hole (24) to a space that creates a vacuum in the ten boxes. In the boxes there are ten pre-formed helical bodies. Aligned and on top of the first and inversely, there is another group of ten boxes and molds that descends and contacts the group that is on the band, making hermetic closure with gaskets (not shown). Both groups move and during the displacement mechanical pressure and vacuum are produced in both groups, which will force the material, which has been treated with emollient, to take the low-relief shape of the mold. Then the set on top raises and moves back to stand on top of the group of ten boxes moving on the band, to start another cycle.

Times and distances are synchronized so that the two groups coincide in each cycle. The cycle time depends on the target production. If it is 2,000 units per minute, the cycle of descending, contacting, displacing the boxes moving with the belt, emptying, raising and moving back to place again on the group moving on the belt, and if in each cycle form ten units, the time will be (2,000/10=200 cycles/minute), in 60 seconds/200 cycles=0.3 seconds per cycle). Negative pressure is exerted for 0.15 seconds, half the cycle time. These cycles and times are currently applied in various production systems.

This process can also be made with rotating cylinders on whose surface there are molds of the helical body. One preformed mold is accommodated in each mold. At each point of tangency, when they come into contact, pressure and vacuum are produced, which are made continuously by rotation and give the final shape.

Step 7—Add agglutinating to each end, dry, then pack.

1. A helical body for cutting, removing and withdrawing parts from a closed or open tubular cavity, or from a solid; that rotates manually or mechanically around the axis of a cross section (2), CHARACTERIZED by a cross section (2) made up of blades (27) attached to a cooperating grip (11) of one or more sections (10), which in its interior has two cooperating ducts, a first injector duct (12) that injects a cooperating element and a second extractor duct (13) through which removed or cut material is withdrawn; where the blades (27) end in a cut point (9) and each blade (27) has a distance (4) between blades that forms a curvature of the internal blade edge (7) and external blade edge (5) that locates shear or remover reinforcements (8) along the helical path; where the helical body is composed of the cooperating shaft (11) or several grip sections (10) with couplings (14) on their axes so that they are oriented and rotate at the same angles and curves as the object or tubular cavity where operates the helical body; It has boxes (28) with holes (25) to fix it to a frame (26), where the box (28) houses half a mold (15) of the helicoid that has blade cavities (16) and injection holes (17) that communicate three sides of the mold (15) with the interior space of the box (28) where the mold (15) is housed.

2. A helical body according to claim 1, CHARACTERIZED in that the cooperating grip (11) is solid or hollow, rigid or flexible and is adhered to the helical body in a rigid or articulated way

3. A helical body according to claim 1, CHARACTERIZED in that the blades (27) that have reinforcements (8) on the cutting edges according to the material and size to be cut, removed, extracted and that can be replaced due to wear or to adapt according to function and material to be cut and removed.

4. A helical body according to claim 1, CHARACTERIZED in that it has internal injector ducts (12) or external extractors (13) along the cooperating grip (11) to inject an element cooperating with the cut and removal and to extract and remove the material.

5. A helical body according to claim 1, CHARACTERIZED in that each section (10) has traction and longitudinal movement and rotation.

6. A helical body according to claim 1, CHARACTERIZED in that each section (10) rotates jointly or independently and at different speeds.

7. A helical body according to claim 1, CHARACTERIZED by a grip (11) at whose ends there is a helical body section of variable diameter, built by pressure and vacuum.

8. A helical body according to claim 1, CHARACTERIZED by the molds (15), which are a half section of the helical body in low relief, aligned one above the other, moving longitudinally and vertically, in a band or cylinder, that contact and by pressure, vacuum and heat on the material, forms helicoidal bodies at each end of the grip.

9. Helicoid body manufacturing process to cut, remove, clean CHARACTERIZED by the following steps:
Step 1—Set up the material from the manufacturer, grip and helicoid material, this one can have in several forms:
1. Material with which the helicoid is manufactured cotton, cellulose or other material in the art, appropriate to manufacture the helicoid body, which can have various forms:
a) flat band 8 to 10 mm wide, 1 to 2 mm thick.
b) inclined plane band 8 to 10 mm wide, where one side is 3 to 4 mm thick and decreases to 1 to 2 mm towards the other side.
c) cylindrical strip, like a thread, with a diameter of 1 to 2 mm.
In any of these forms the material is pre-cut or continuous. In this case the preforming step includes the cutting of each part when spinning.
2. Preformed grip, made of impregnated paper, cellulose, wood, 50 to 60 mm long and 2 to 3 mm in diameter. It can have different lengths and diameters depending on the application for which it is intended.
Step 2—Add emollient to each end of the grip to help giving and keeping shape, remove fiber particles that can come off inside the ear, by dispersion in cold, hot, steam, about 0.025 ml.
Step 3—Preform the helicoid body by wrapping 0.5 to 1 gram of cotton, cellulose, or other material with suitable characteristics (soft, absorbent, non-particles) around the two ends of the grip.
This winding results in a conical helical body with diameters at the base of 5 to 6 mm and at the upper end of 2.5 to 5 mm, which are 25 to 30% larger than the final size of the helical body for cleaning wax. These measures are modified depending on the application to which it is intended. The pre-shape can be made in several ways:
a) Place the grip (11) in a band or cylinder, which has a cavity with its shape and means to make it rotate.
b) Mold a flat band of material such as cotton in the middle of a longitudinally cut mold that has the shape of the helicoid in low relief, hold independently of the grip (11). This half of the mold is engraved in a block 20 to 23 mm square and 6 to 13 mm high and varies according to the application for which it is intended.
The movement of the band or the cylinder causes the material to be rolled to come closer and remains in contact with each end of the grip (11) and in proximity to the cavity that has the half mold of the helicoid engraved in low relief. At the ends of the grip (11) the material, such as cotton, is hold, rotated and rolled over the half of the mold, forcing it to take the shape of the half mold of the helicoid body. At each end 0.02 to 0.03 grams of the material are wound. The movement of the band or the cylinder brings the pre-formed helical body to a storage space.
c) Roll up a cylindrical strip of material at each end of the grip, giving more winding time at the base of the cone and moving towards the smaller diameter. A cone-roll with more material at the bottom will result.
d) Mold a cylindrical strip by rolling against half a mold while the material source moves longitudinally to the axis of the mold.
Step 4—Place the preformed helicoid bodies in a container that is close to the band or cylinder that gives the final shape.
Step 5—Arrange the preformed helicoid bodies in the grip that is on a band and that contains the halves of the mold of ten helicoid bodies, band that moves as it is currently done in the art.
Step 6—Give final shape in cycles. Ten boxes with half helical body mold and grip are accommodated in a frame on a moving belt. The grip has in its lower part a connecting hole (24) to a space that creates a vacuum in the ten boxes. In the boxes there are ten pre-formed helical bodies. Aligned and on top of the first and inversely, there is another group of ten boxes and molds that descends and contacts the group that is on the band, making hermetic closure with gaskets (not shown). Both groups move and during the displacement mechanical pressure and vacuum are produced in both groups, which will force the material, which has been treated with emollient, to take the low-relief shape of the mold. Then the set on top raises and moves back to stand on top of the group of ten boxes moving on the band, to start another cycle.
Times and distances are synchronized so that the two groups coincide in each cycle. The cycle time depends on the target production. If it is 2,000 units per minute, the cycle of descending, contacting, displacing the boxes moving with the belt, emptying, raising and moving back to place again on the group moving on the belt, and if in each cycle form ten units, the time will be (2,000/10=200 cycles/minute), in 60 seconds/200 cycles=0.3 seconds per cycle). Negative pressure is exerted for 0.15 seconds, half the cycle time. These cycles and times are currently applied in various production systems.
This process can also be made with rotating cylinders on whose surface there are molds of the helical body. One preformed mold is accommodated in each mold. At each point of tangency, when they come into contact, pressure and vacuum are produced, which are made continuously by rotation and give the final shape.
Step 7—Add agglutinating to each end, dry, then pack.
